(19) United States
(12) Patent Application Publication
(10) Pub. No.: US 2022/0218406 A1
(43) Pub. Date: Jul. 14, 2022

(54) SYSTEM AND METHOD FOR CONTROLLED GRASPING AND ENERGY DELIVERY

(71) Applicant: INTUITIVE SURGICAL OPERATIONS, INC., Sunnyvale, CA (US)

(72) Inventors: Adrit LATH, Palo Alto, CA (US); Jason HEMPHILL, Los Gatos, CA (US); Iman KHALAJI, Santa Clara, CA (US); Scott E. MANZO, Shelton, CT (US); Duane W. MARION, Scottsdale, AZ (US); Mathew NUSSBAUM, Campbell, CA (US); Adam ROSS, Prospect, CT (US); Jignesh SHAH, Sunnyvale, CA (US); Benjamin J. SCHOETTGEN, Los Gatos, CA (US)

(21) Appl. No.: 17/610,411

(22) PCT Filed: May 8, 2020
(86) PCT No.: PCT/US2020/032037

§ 371 Date: Nov. 10, 2021

Related U.S. Application Data
(60) Provisional application No. 62/846,387, filed on May 10, 2019.

(51) Int. Cl. A61B 18/14; A61B 34/30; A61B 34/10

(52) U.S. Cl. CPC A61B 18/1442; A61B 2018/00601; A61B 34/10; A61B 34/30

(57) ABSTRACT

Systems and methods of controlled grasping and energy delivery include a computer-assisted device. The computer-assisted device includes an end effector and one or more processors. The end effector includes a first jaw, a second jaw, and a plurality of electrodes for delivering energy. The one or more processors are configured to grasp a material using the first jaw and the second jaw, determine characteristics of the grasp, determine characteristics of the material, and control one or more of the grasp or energy delivery by the plurality of electrodes based on the determined characteristics of the grasp and the determined characteristics of the material. According to some embodiments, the characteristics of the material include one or more of thermal, dielectric, or stiffness of the material. In some embodiments, the characteristics of the grasp include one or more of applied pressure, jaw angle, jaw separation, force, torque, or wrist articulation.

### RELATED APPLICATION

This application claims the benefit to U.S. Provisional Application No. 62/846,387, filed May 10, 2019, which is incorporated by reference herein.

### TECHNICAL FIELD

The present disclosure relates generally to operation of devices with end effectors and more particularly to operation of end effectors capable of grasping material and applying energy to the grasped material.

### BACKGROUND

More and more devices are being replaced with computer-assisted electronic devices. This is especially true in industrial, entertainment, educational, and other settings. As a medical example, the hospitals of today with large arrays of electronic devices being found in operating rooms, interventional suites, intensive care wards, emergency rooms, and/or the like. For example, glass and mercury thermometers are being replaced with electronic thermometers, intravenous drip lines now include electronic monitors and flow regulators, and traditional hand-held surgical and other medical instruments are being replaced by computer-assisted medical devices.

These computer-assisted devices are useful for performing operations and/or procedures on materials, such as the tissue of a patient. With many computer-assisted devices, an operator, such as a surgeon and/or other medical personnel, may typically manipulate input devices using one or more controls on an operator console. As the operator operates the various controls at the operator console, the commands are relayed from the operator console to a computer-assisted device located in a workspace where they are used to position and/or actuate one or more end effectors and/or tools that are mounted (e.g., via repositionable arms) to the computer-assisted device. In this way, the operator is able to perform one or more procedures on material in the workspace using the end effectors and/or tools. Depending upon the desired procedure and/or the tools in use, the desired procedure may be performed partially or wholly under control of the operator using teleoperation and/or under semi-autonomous control where the computer-assisted device may perform a sequence of operations based on one or more activation actions by the operator.

Computer-assisted devices, whether actuated manually, teleoperatively, and/or semi-autonomously may be used in a variety of operations and/or procedures and may have various configurations. Many such instruments include an end effector mounted at a distal end of a shaft that may be mounted to the distal end of a repositionable or articulated arm. In many operational scenarios, the shaft may be configured to be inserted into the workspace via an opening in the workspace. As a medical example, the shaft may be inserted (e.g., laparoscopically, thoracoscopically, and/or the like) through an opening (e.g., a body wall incision, a natural orifice, and/or the like) to reach a remote surgical site. In some instruments, an articulating wrist mechanism may be mounted to the distal end of the instrument's shaft to support the end effector with the articulating wrist providing the ability to alter an orientation of the end effector relative to a longitudinal axis of the shaft.

End effectors of different design and/or configuration may be used to perform different tasks, procedures, and functions so as to be allow the operator to perform any of a variety of procedures on a material. Examples include, but are not limited to, cauterizing, ablating, suturing, cutting, stapling, fusing, sealing, etc., and/or combinations thereof. Accordingly, end effectors can include a variety of components and/or combinations of components to perform these procedures.

In many embodiments, the size of the end effector is typically kept as small as possible while still allowing it to perform its intended task. One approach to keeping the size of the end effector small is to accomplish actuation of the end effector through the use of one or more inputs at a proximal end of the tool, which is typically located externally and/or peripherally to the workspace. Various gears, levers, pulleys, cables, rods, bands, and/or the like, may then be used to transmit actions from the one or more inputs along the shaft of the tool and to actuate the end effector. In some embodiments, a transmission mechanism at the proximal end of the tool interfaces with various motors, solenoids, servos, active actuators, hydraulics, pneumatics, and/or the like provided on a repositionable arm of the computer-assisted device. The motors, solenoids, servos, active actuators, hydraulics, pneumatics, and/or the like typically receive control signals through a master controller and provide input in the form of force and/or torque at the proximal end of the transmission mechanism, which the various gears, levers, pulleys, cables, rods, bands, and/or the like ultimately transmit to actuate the end effector at the distal end of the transmission mechanism.

Additionally, in many embodiments, the tools and/or end effectors may include one or more energy delivery components that may be used to deliver ultrasonic, radio frequency, electrical, magnetic, thermal, light, and/or other energies to the material grasped by and/or in proximity to the end effector. In some embodiments, the end effectors may include one or more sensors for monitoring the energy delivery. Various wires, cables, optical fibers, and/or like may be used to deliver the energy to end effector from a control module located proximal to the end effector (e.g., in a control console) and/or provide the sensor information to the control module.

Because of the remote nature of the operation of such end effectors, it may be difficult in some cases for the operator to directly monitor the end effector and/or the energy delivery to the material. For example, in some cases, other portions of the computer-assisted device, including the end effector itself, and/or other materials and/or items in the workspace may hide from view some or all of the end effector during its operation.

Accordingly, improved methods and systems for the operation of computer-assisted devices, such as computer-assisted devices having end effectors used to grasp and/or deliver energy to a material are desirable. In some examples, it may be desirable to provide automated control of the computer-assisted device and/or the end effectors so as to help ensure that the tool may be able to successfully perform a desired procedure on the material.

### SUMMARY

Consistent with some embodiments, a computer-assisted device includes an end effector and one or more processors. The end effector includes a first jaw, a second jaw, and a plurality of electrodes for delivering energy. The one or more processors are configured to grasp a material using the first jaw and the second jaw, determine one or more characteristics of the grasp, determine one or more characteristics of the material, and control one or more of the grasp or energy delivery by the plurality of electrodes based on the determined one or more characteristics of the grasp and the determined one or more characteristics of the material.

Consistent with some embodiments, a method includes grasping, by one or more processors, a material using a first jaw and a second jaw of an end effector; determining, by the one or more processors, one or more characteristics of the grasp; determining, by the one or more processors, one or more characteristics of the material; and controlling, by the one or more processors, one or more of the grasping or energy delivery by a plurality of electrodes of the end effector based on the determined one or more characteristics of the grasp and the determined one or more characteristics of the material.

Consistent with some embodiments, a non-transitory machine-readable medium comprising a plurality of machine-readable instructions which when executed by one or more processors are adapted to cause the one or more processors to perform any of the methods described herein.

### BRIEF DESCRIPTION OF THE DRAWINGS

FIG. 1 is a simplified diagram of a computer-assisted system according to some embodiments.

FIG. 2 is a simplified diagram showing a tool suitable for use with the computer-assisted system of FIG. 1 according to some embodiments.

FIGS. 3A and 3B are simplified side and top views of a jaw of a tool according to some embodiments.

FIG. 4 is a simplified diagram of a method for grasping and energy delivery according to some embodiments.

FIG. 5 is a simplified diagram of a method for energy delivery according to some embodiments.

In the figures, elements having the same designations have the same or similar functions.

### DETAILED DESCRIPTION

This description and the accompanying drawings that illustrate inventive aspects, embodiments, implementations, or modules should not be taken as limiting—the claims define the protected invention. Various mechanical, compositional, structural, electrical, and operational changes may be made without departing from the spirit and scope of this description and the claims. In some instances, well-known circuits, structures, or techniques have not been shown or described in detail in order not to obscure the invention. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, this description's terminology is not intended to limit the invention. For example, spatially relative terms-such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, implementation, or module may, whenever practical, be included in other embodiments, implementations, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

In some instances, well known methods, procedures, components, and circuits have not been described in detail so as not to unnecessarily obscure aspects of the embodiments.

This disclosure describes various devices, elements, and portions of computer-assisted devices and elements in terms of their state in three-dimensional space. As used herein, the term “position” refers to the location of an element or a portion of an element in a three-dimensional space (e.g., three degrees of translational freedom along Cartesian x-, y-, and z-coordinates). As used herein, the term “orientation” refers to the rotational placement of an element or a portion of an element (three degrees of rotational freedom—e.g., roll, pitch, and yaw). As used herein, the term “shape” refers to a set positions or orientations measured along an element. As used herein, and for a device with repositionable arms, the term “proximal” refers to a direction toward the base of the computer-assisted device along its kinematic chain and “distal” refers to a direction away from the base along the kinematic chain.

Aspects of this disclosure are described in reference to computer-assisted systems and devices, which may include systems and devices that are teleoperated, remote-controlled, autonomous, semiautonomous, robotic, and/or the like. Further, aspects of this disclosure are described in terms of an implementation using a surgical system, such as the da Vinci® Surgical System commercialized by Intuitive Surgical, Inc. of Sunnyvale, Calif. Knowledgeable persons will understand, however, that inventive aspects disclosed herein may be embodied and implemented in various ways, including robotic and, if applicable, non-robotic embodiments and implementations. Implementations on da Vinci® Surgical Systems are merely exemplary and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (without return to a human or animal anatomy) and for procedures on human or animal cadavers. Further, these techniques can also be used for medical treatment or diagnosis procedures that include, or do not include, surgical aspects.

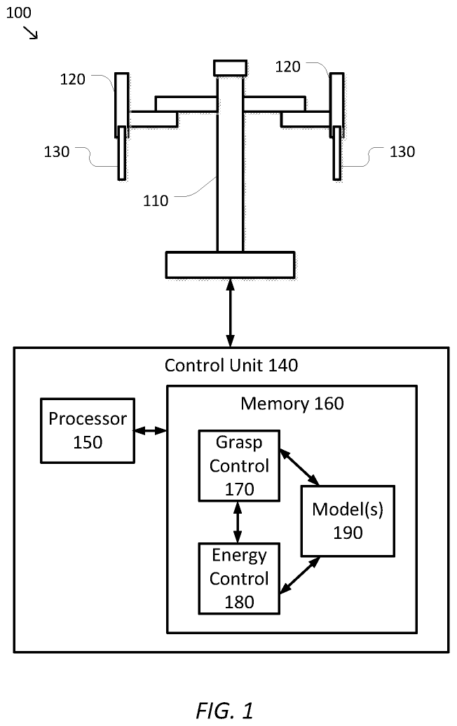

FIG. 1 is a simplified diagram of a computer-assisted system 100 according to some embodiments. As shown in FIG. 1, computer-assisted system 100 includes a device 110 with one or more repositionable arms 120. Each of the one or more repositionable arms 120 may support one or more tools 130. In some examples, device 110 may be consistent with a computer-assisted medical device. The one or more tools 130 may include tools, imaging devices, and/or the like. In some medical examples, the tools may include medical tools, such as clamps, grippers, retractors, cautery tools, suction tools, suturing devices, and/or the like. In some medical examples, the imaging devices may include endoscopes, cameras, ultrasonic devices, fluoroscopic devices, and/or the like. In some examples, each of the one or more tools 130 may be inserted into a workspace (e.g., anatomy of a patient, a veterinary subject, and/or the like) through a respective cannula mounted to a respective one of the one or more repositionable arms 120. In some examples, a direction of view of an imaging device may correspond to an insertion axis of the imaging device and/or may be at an angle relative to the insertion axis of the imaging device. In some examples, each of the one or more tools 130 may include an end effector that may be capable of both grasping a material (e.g., tissue of a patient) located in the workspace and delivering energy to the grasped material. In some examples, the energy may include ultrasonic, radio frequency, electrical, magnetic, thermal, light, and/or the like. In some embodiments, computer-assisted system 100 may be found in an operating room and/or an interventional suite.

Device 110 is coupled to a control unit 140 via an interface. The interface may include one or more cables, connectors, and/or buses and may further include one or more networks with one or more network switching and/or routing devices. Control unit 140 includes a processor 150 coupled to memory 160. Operation of control unit 140 is controlled by processor 150. And although control unit 140 is shown with only one processor 150, it is understood that processor 150 may be representative of one or more central processing units, multi-core processors, microprocessors, microcontrollers, digital signal processors, field programmable gate arrays (FPGAs), application specific integrated circuits (ASICs), graphics processing units (GPUs), tensor processing units (TPUs), and/or the like in control unit 140. Control unit 140 may be implemented as a stand-alone subsystem and/or as a board added to a computing device or as a virtual machine.

Memory 160 may be used to store software executed by control unit 140 and/or one or more data structures used during operation of control unit 140. Memory 160 may include one or more types of machine readable media. Some common forms of machine readable media may include floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

As shown, memory 160 includes a grasp control module 170, an energy control module 180, and one or more models 190, that may be used to control and/or monitor one of the one or more tools 130 of device 110 as is described in further detail below. And although FIG. 1 shows grasp control module 170, energy control module 180, and the one or more models 190 as separate elements stored within a same memory 160 of a same control unit 140, other configurations are possible. In some examples, grasp control module 170, energy control module 180, and the one or more models 190 may be combined partially and/or completely within a same module. In some examples, grasp control module 170, energy control module 180, and the one or more models 190 may alternatively be stored in different memories, and/or associated with different control units. Further, even though grasp control module 170, energy control module 180, and the one or more models 190 are characterized as software modules, each may be implemented using software, hardware, and/or a combination of hardware and software.

In some embodiments, grasp control module 170 is responsible for managing the mechanical operation of the one or more tools 130. In some examples, grasp control module 170 may monitor one or more sensors (e.g., one or more encoders, potentiometers, fiber optic sensors, and/or the like) used to track the position, orientation, articulation, and/or mechanical actuation of the one or more tools 130 and their respective end effectors and/or one or more material properties of material being interacted with by the one or more tools 130 and their respective end effectors. In some examples, grasp control module 170 may control the position, orientation, articulation, and/or mechanical actuation of the one or more tools 130 and their respective end effectors using one or more actuators based on the monitoring and/or the one or more models 190. In some examples, control of the position, orientation, articulation, and/or mechanical actuation of the one or more tools 130 and their respective end effectors may include controlling one or more degrees of freedom including, as examples, an insertion depth, a roll, a pitch, a yaw, a wrist articulation, an angle between jaws, a force or torque applied, an amount of cutting and/or transaction using a moveable element, an amount of stapling, and/or the like.

In some embodiments, energy control module 180 is responsible for managing the energy delivery operation of the one or more tools 130. In some examples, energy control module 180 may monitor one or more sensors used to track the energy delivered by the one or more tools 130 and their respective end effectors and/or one or more material properties of material being interacted with by the one or more tools 130 and their respective end effectors. In some examples, energy control module 180 may control the energy delivered by the one or more tools 130 and their respective end effectors using one or more transducers, signal generators, and/or the like based on the monitoring and/or the one or more models 190.

In some embodiments, the one or more models 190 include models used by grasp control module 170 and/or energy control module 180 to control mechanical and/or energy delivery, respectively, of the one or more tools 130 and their respective end effectors. In some examples, the one or more models 190 may include one or more kinematic models, one or more material models, and/or one or more prediction models used to provide recommendations regarding mechanical control and/or energy delivery by the one or more tools 130 and their respective end effectors as is described in further detail below. In some examples, the one or more models may include one or more functions, one or more look up tables, one or more maps, one or more parameterized curves, one or more machine learning models (e.g., one or more neural networks), and/or the like. In some examples, the one or more parameterized curves may include linear relationships, piece-wise linear relationships, quadratic relationships, higher-order relationships, and/or the like determined via curve fitting, regression, and/or the like from data collected from previous grasp and/or energy delivery applications.

As discussed above and further emphasized here, FIG. 1 is merely an example which should not unduly limit the scope of the claims. One of ordinary skill in the art would recognize many variations, alternatives, and modifications. According to some embodiments, computer-assisted system 100 may include any number of computer-assisted devices with articulated arms and/or instruments of similar and/or different in design from computer-assisted device 110. In some examples, each of the computer-assisted devices may include fewer or more articulated arms and/or instruments.

According to some embodiments, the arrangement of grasp control module 170, energy control module 180, and/or the one or more models 190 may be different than as depicted in FIG. 1. In some examples, grasp control module 170, energy control module 180, and/or the one or more models 190 may be distributed across more than one control unit. In some examples, grasp control module 170 and energy control module 180 may be included in a single control module. In some examples, the one or more models 190 may be included in grasp control module 170 and/or energy control module 180.

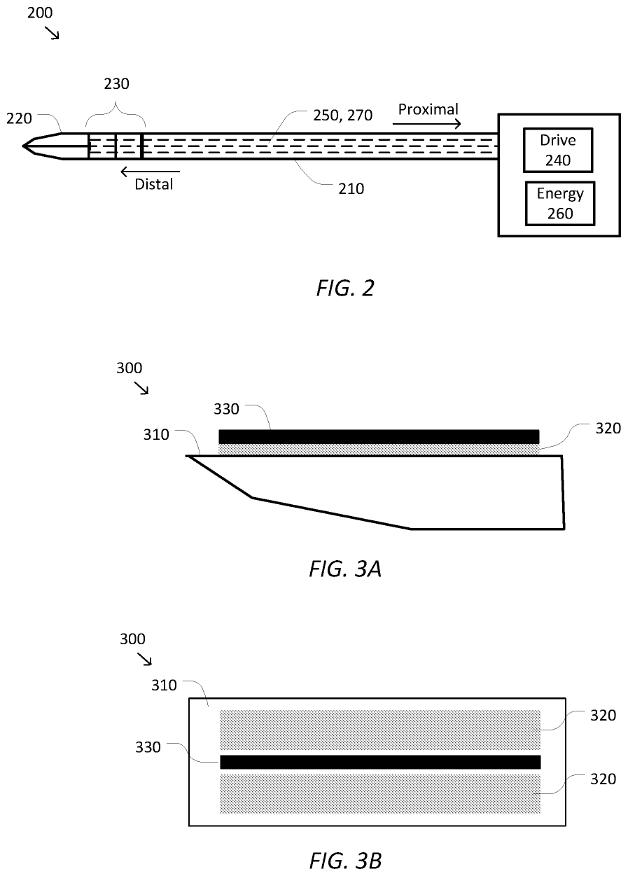

FIG. 2 is a simplified diagram showing a tool 200 suitable for use with the computer-assisted system 100 according to some embodiments. In some embodiments, tool 200 may be consistent with any of the tools 130 of FIG. 1. The directions “proximal” and “distal” as depicted in FIG. 2 and as used herein help describe the relative orientation and location of components of tool 200.

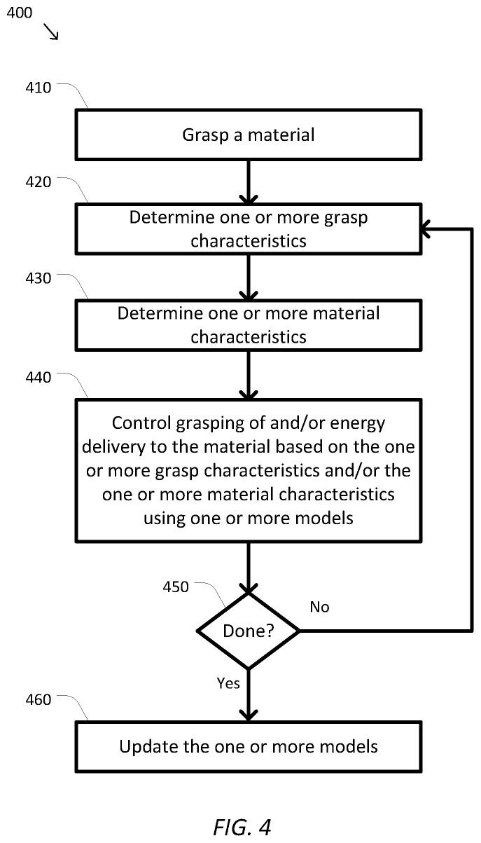

As shown in FIG. 2, tool 200 includes a long shaft 210 used to couple an end effector 220 located at a distal end of shaft 210 to where the tool 200 is mounted to a repositionable arm and/or a computer-assisted device at a proximal end of shaft 210. Depending upon the particular procedure for which the tool 200 is being used, shaft 210 may be inserted through an opening (e.g., a body wall incision, a natural orifice, and/or the like) in order to place end effector 220 in proximity to a workspace, such as a remote surgical site located within the anatomy of a patient. As further shown in FIG. 2, end effector 220 is generally consistent with a two-jawed gripper-style end effector, which in some embodiments may further include an energy delivery mechanism as is described in further detail below with respect to FIGS. 3A, 3B, and 4. However, one of ordinary skill would understand that different tools 200 with different end effectors 220 are possible and may be consistent with the embodiments of tool 200 as described elsewhere herein.

A tool, such as tool 200 with end effector 220 typically relies on multiple degrees of freedom (DOFs) during its operation. Depending upon the configuration of tool 200 and the repositionable arm and/or computer-assisted device to which it is mounted, various DOFs that may be used to position, orient, and/or operate end effector 220 are possible. In some examples, shaft 210 may be inserted in a distal direction and/or retreated in a proximal direction to provide an insertion DOF that may be used to control how deep within the workspace end effector 220 is placed. In some examples, shaft 210 may be able rotate about its longitudinal axis to provide a roll DOF that may be used to rotate end effector 220. In some examples, additional flexibility in the position and/or orientation of end effector 220 may be provided by an articulated wrist 230 that is used to couple end effector 220 to the distal end of shaft 210. In some examples, articulated wrist 230 may include one or more rotational joints, such as one or more roll, pitch or yaw joints that may provide one or more “roll,” “pitch,” and “yaw” DOF(s), respectively, that may be used to control an orientation of end effector 220 relative to the longitudinal axis of shaft 210. In some examples, the one or more rotational joints may include a pitch and a yaw joint; a roll, a pitch, and a yaw joint, a roll, a pitch, and a roll joint; and/or the like. In some examples, end effector 220 may further include a grip DOF used to control the opening and closing of the jaws of end effector 220. Depending upon the configuration, end effector 220 may include two moveable jaws that are articulated with respect to each other about a hinge point located near a proximal end of end effector 220 or one fixed jaw and one moveable jaw that is articulated with respect to the fixed jaw about the hinge point. In some examples, the two moveable jaws may include two parallel jaw faces whose distance there between is adjusted, such as by using one or more cams, to open and close the jaws.

Tool 200 further includes a drive system 240 located at the proximal end of shaft 210. Drive system 240 includes one or more components for introducing forces and/or torques to tool 200 that may be used to manipulate the various DOFs supported by tool 200. In some examples, drive system 240 may include one or more motors, solenoids, servos, active actuators, hydraulic actuators, pneumatic actuators, and/or the like that are operated based on signals received from a control unit, such as control unit 140 of FIG. 1. In some examples, drive system 240 may manipulate a subset of the various DOFs with others of the various DOFs being, for examples, controlled manually by an operator. In some examples, the signals may include one or more currents, voltages, pulse-width modulated wave forms, and/or the like. In some examples, drive system 240 may include one or more shafts, gears, pulleys, rods, bands, and/or the like which may be coupled to corresponding motors, solenoids, servos, active actuators, hydraulics, pneumatics, and/or the like that are part of the articulated arm, such as any of the repositionable arms 120, to which tool 200 is mounted. In some examples, the one or more drive inputs, such as shafts, gears, pulleys, rods, bands, and/or the like, may be used to receive forces and/or torques from the motors, solenoids, servos, active actuators, hydraulics, pneumatics, and/or the like and apply those forces and/or torques to adjust the various DOFs of tool 200.

In some embodiments, the forces and/or torques generated by and/or received by drive system 240 may be transferred from drive system 240 and along shaft 210 to the various joints and/or elements of tool 200 located distal to drive system 240 using one or more drive mechanisms 250. In some examples, the one or more drive mechanisms may include one or more gears, levers, pulleys, cables, rods, bands, and/or the like. In some examples, shaft 210 is hollow and drive mechanisms 250 pass along the inside of shaft 210 from drive system 240 to the corresponding DOF in end effector 220 and/or articulated wrist 230. In some examples, each of drive mechanisms 250 may be a cable disposed inside a hollow sheath or lumen in a Bowden cable like configuration, a shaft or rod whose rotation actuates a corresponding DOF, and/or the like. In some examples, the cable and/or the inside of the lumen may be coated with a low-friction coating such as polytetrafluoroethylene (PTFE) and/or the like. In some examples, as the proximal end of each of the cables is pulled and/or pushed inside drive system 240, such as by wrapping and/or unwrapping the cable about a capstan or shaft, the distal end of the cable moves accordingly and applies a suitable force and/or torque to adjust one of the DOFs of end effector 220, articulated wrist 230, and/or tool 200. In some examples, drive system 240 may be controlled and/or receive instructions from a grasp control module, such as grasp control module 170.

In some embodiments, tool 200 further includes an energy system 260 located at the proximal end of shaft 210. Energy system 260 includes one or more components for generating energy for delivery by tool 200. In some examples, the energy may be in one or more energy modalities including ultrasonic, radio frequency, electrical, magnetic, thermal, light, and/or the like. In some examples, energy system 260 may include one or more transducers, signal generators, and/or the like that are operated based on signals received from a control unit, such as control unit 140 of FIG. 1. In some examples, the signals may include one or more currents, voltages, pulse-width modulated wave forms, light patterns, and/or the like.

In some embodiments, the energy generated by and/or received by energy system 260 may be transferred from energy system 260 and along shaft 210 to the various joints and/or elements of tool 200 located distal to energy system 260 using one or more energy delivery mechanisms 270. In some examples, the one or more energy mechanisms may include one or more wires, cables, optical fibers, and/or like. In some examples, shaft 210 is hollow and energy delivery mechanisms 270 pass along the inside of shaft 210 from energy system 260 to end effector 220 for delivery to a material within the workspace. In some examples, energy system 260 may be controlled and/or receive instructions from an energy control module, such as energy control module 180.

FIGS. 3A and 3B are simplified side and top views of a jaw 300 of a tool according to some embodiments. In some embodiments, jaw 300 is consistent with either or both of the jaws of end effector 220. As shown in FIGS. 3A and 3B, jaw 300 includes a jaw face 310, one or more sealing electrodes 320, and one or more cutting electrodes 330. In some examples, jaw face 310 is generally planar and is parallel with a jaw face of an opposing jaw when jaw 300 is closed relative to the opposing jaw and at an angle to the jaw face of the opposing jaw when jaw 300 is open relative to the opposing jaw. As shown, the one or more sealing electrodes 320 are generally located along the outsides of jaw 300 and the one or more cutting electrodes are located generally along the center line of jaw 300. In some examples, this configuration allows the one or more sealing electrodes 320 to seal the two ends of a material that are separated when the one or more cutting electrodes 330 are used to cut the material. In addition, each of the one or more sealing electrodes 320 and the one or more cutting electrodes 330 are generally aligned with one or more corresponding sealing electrodes and one or more corresponding cutting electrodes, respectively, on the opposing jaw. Additional examples of possible arrangements for jaw 300, sealing electrodes 320, and cutting electrodes 330 are described in further detail in commonly-owned U.S. Pat. No. 9,055,961 disclosing “Fusing and Cutting Surgical Instrument and Related Methods” and commonly-owned International Patent Application No. PCT/US2018/39912 disclosing “Electrosurgical Instrument with Compliant Elastomeric Electrode,” both of which are incorporated by reference.

According to some embodiments, the ability of a pair of electrodes (e.g., between an electrode on one jaw and an electrode on the opposing jaw) to cut and/or seal may be controlled by applying an appropriate voltage differential between the pair of electrodes. In some examples, a first voltage differential for cutting may be different than a second voltage potential for sealing. In some examples, the voltage differentials for cutting and/or sealing are selected based on the material to be cut and/or sealed. In some examples, when the material is anatomical tissue, a voltage differential in a range of about 250 V to 400 V may generally cause cutting and a voltage differential in a range of about 50 V to 150 V may generally cause sealing. In some examples, a voltage differential between the cutting voltage differential and the sealing voltage differential may result in a combination of cutting and sealing. In some examples, an amount of energy delivered by a pair of electrodes may further be controlled by limiting current flow between the pair of electrodes by, for example, use of an appropriate current limiter. In some examples, an energy system, such as energy system 260 may be used to control the voltage differential and current limits applied to the pair of electrodes. In some examples, the energy may be delivered as a series of energy pulses by controlling the voltage differently and/or the current as a series of pulses.

In some embodiments, despite their description as sealing electrodes and/or cutting electrodes, both the one or more sealing electrodes 320 and/or the one or more cutting electrodes 330 may be used for both sealing and/or cutting by controlling the voltage differential between the one or more sealing electrodes 320 and the one or more corresponding sealing electrodes in the opposing jaw and/or the one or more cutting electrodes 330 and the one or more corresponding cutting electrodes in the opposing jaw. In some examples, cutting may be performed by applying cutting energy between a cutting electrode 330 on one of the jaws and one or more of the sealing electrodes 320 on the opposing jaw.

As discussed above and further emphasized here, FIGS. 3A and 3B are merely examples which should not unduly limit the scope of the claims. One of ordinary skill in the art would recognize many variations, alternatives, and modifications. According to some embodiments, a relative size of the surface areas of the one or more sealing electrodes 320 and/or the one or more cutting electrodes 330 may be different than as depicted in FIG. 3B. In some examples, each of the one or more sealing electrodes 320 may have a same or smaller surface area than the one or more cutting electrodes 330. According to some embodiments, the relative height that the one or more sealing electrodes 320 and/or the one or more cutting electrodes 330 protrudes above jaw face 310 may be different than as depicted in FIG. 3A. In some examples, one or more of the one or more sealing electrodes 320 and/or the one or more cutting electrodes 330 may be flush with jaw face 310 and/or recessed below jaw face 310. In some examples, a relative height of the one or more sealing electrodes may be the same and/or shorter than the height of the one or more cutting electrodes. According to some embodiments, an axial length (from proximal to distal) along jaw 300 of each of the one or more sealing electrodes 320 and/or the one or more cutting electrodes 330 may be longer, shorter, and/or of different lengths.

According to some embodiments, control of an end effector, such as end effector 220, that supports both grasping (e.g., using opposing jaws) and energy delivery (e.g., using the one or more sealing electrodes 320 and/or the one or more cutting electrodes 330) are typically controlled using separate systems. For example, a drive system and a corresponding grasp control module may control the grasping while an energy system and a corresponding energy control module may control the energy delivery. In some examples, there may be little or no cooperation between drive system/grasp control module and the energy system/energy control module. That is, the drive system/grasp control module may control grasping based on mechanical and/or kinematic properties of the grasped material and not the electrical properties of the grasped material that indicate whether sealing and/or cutting are occurring satisfactorily. Similarly, the energy system/energy control module may control energy delivery based on electrical properties of the grasped material and not the mechanical and/or kinematic properties of the material that indicate whether a grasp of the material that is likely to result in good sealing and/or cutting has been obtained. Accordingly, better sealing and cutting of a grasped material may be obtained when the drive system/grasp control module and the energy system/energy control module work together to control both the grasping and energy delivery so that both the grasping and energy delivery work to complement each other.

FIG. 4 is a simplified diagram of a method 400 for grasping and energy delivery according to some embodiments. One or more of the processes 410-460 of method 400 may be implemented, at least in part, in the form of executable code stored on non-transitory, tangible, machine readable media that when run by one or more processors (e.g., the processor 150 in control unit 140) may cause the one or more processors to perform one or more of the processes 410-460. In some embodiments, method 400 may be performed by one or more modules, such as grasp control module 170 and/or energy control module 180. In some embodiments, portions of method 400 associated with grasping (e.g., sensing of mechanical and/or kinematic information and mechanical control of grasping jaws) may be performed by grasp control module 170 and portions of method 400 associated with energy delivery (e.g., sensing of electrical properties and controlling of energy delivery) may be performed by energy control module 180 with grasp control module 170 and energy control module 180 cooperating to share sensor and control information so as to optimize energy delivery to a grasped material. In some embodiments, process 460 is optional and may be omitted. In some embodiments, method 400 may be performed in a different order than the order implied by FIG. 4. In some examples, processes 430 may be performed before process 420 and/or processes 420 and/or 430 may be performed concurrently. In some examples, process 420 and 430 may be performed concurrently with process 440.

At a process 410, a material is grasped. In some examples, the material may be grasped between the jaws of an end effector, such as end effector 220. In some examples, each of the jaws may be consistent with jaw 300. In some examples, the material may be grasped using a drive system, such as drive system 240, under the control of a grasp control module, such as grasp control module 170. In some examples, the grasp may occur based on a command received from an operator. In some examples, the grasp may include actuation of the jaws until a desired angle between the jaws is reached, a desired separation between the jaws is reached, and/or a desired force or torque limit indicating a desired grasp strength is reached. In some examples, the grasp may actuate the jaws to a desired position set point (e.g., a desired angle and/or separation between the jaws) subject to an upper force and/or torque limit. In some examples, the force or torque limits may be implemented as a current limit on the one or more actuators used to actuate the jaws.

At a process 420, one or more grasp characteristics are determined. In some examples, the one or more grasp characteristics may include an applied pressure by which the material is being grasped and/or a rate of change in the applied pressure. In some examples, the applied pressure may be determined using one or more pressure sensors (e.g., one or more strain gauges, pressure transducers, pressure sensitive fiber optic sensors, and/or the like) located along the face of one or both of the jaws. In some examples, the rate of change in applied pressure may be determined using a numerical differentiation technique (e.g., using the divided difference method) from two or more applied pressure readings obtained over time. In some examples, the applied pressure may be determined indirectly from one or more other grasp characteristics.

In some examples, the one or more grasp characteristics may include a measurement of current jaw angle (or separation) and/or a rate of change in jaw angle (or separation) obtained from one or jaw angle (or separation) sensors. In some examples, the rate of change in jaw angle (or separation) may be determined using a numerical differentiation technique (e.g., using the divided difference method) from two or more jaw angle (or separation) readings obtained over time.

In some examples, the one or more grasp characteristics may include an applied force and/or torque and/or a rate of change in applied force and/or torque as applied by the jaws to the grasped material obtained from one or more force and/or torque sensors associated with the jaws and/or the one or more actuators used to actuate the jaws. In some examples, the rate of change in applied force and/or torque may be determined using a numerical differentiation technique (e.g., using the divided difference method) from two or more force and/or torque readings obtained over time. In some examples, the force and/or torque may be determined based on one or more currents used to actuate the one or more actuators used to actuate one or both of the jaws.

In some examples, the one or more grasp characteristics may include additional kinematic information associated with the tool and/or the end effector whose jaws are used to grasp the material. In some examples, the additional kinematic information may include information about an amount and/or a type of articulation of an articulated wrist (e.g., articulated wrist 230) of the tool.

In some examples, the one or more grasp characteristics may be determined from one or more images obtained from an imaging device of the jaws and the grasped material. In some examples, the one or more images may be used to measure jaw angle, jaw separation, and/or wrist articulation. In some examples, the imaging device may be an endoscope and/or a stereo endoscope. In some examples, the imaging device may be mounted as a tool to a repositionable arm, such as one of the one or more repositionable arms 120.

At a process 430, one or more material characteristics are determined. In some examples, the one or more material characteristics may include a temperature of the grasped material and/or a rate of change in the temperature. In some examples, the temperature of the material may be determined using one or more temperature sensors, such as one or more thermocouples, thermal resistors, and/or the like, located only the face of one or both of the jaws. In some examples, the temperature or other thermal properties of the material may be determined by delivering non-therapeutic energy to the material. In some examples, the temperature of the grasped material may be determined using an infrared sensor, such as an infrared sensor mounted on an imaging device, directed toward the jaws and the grasped material. In some examples, the rate of change in temperature may be determined using a numerical differentiation technique (e.g., using the divided difference method) from two or more temperature readings obtained over time. In some examples, the temperature of the grasped material may be determined indirectly from one or more of the grasp characteristics and/or one or more of the other material characteristics.

In some examples, the one or more material characteristics may include an impedance of the grasped material and/or a rate of change in impedance of the grasped material obtained by measuring electrical characteristics between one or more pairs of sealing and/or cutting electrodes used to seal and/or cut the grasped material. In some examples, each of the one or more pairs of sealing and/or cutting electrodes may include one of the one more sealing electrodes 320 on jaw 300 and a corresponding sealing electrode on an opposing jaw and/or one of the one or more cutting electrodes 330 on jaw 300 and a corresponding cutting electrode on an opposing jaw. In some examples, the rate of change in impedance may be determined using a numerical differentiation technique (e.g., using the divided difference method) from two or more impedance (or separation) readings obtained over time.

In some examples, the one or more material characteristics may include a stiffness of the grasped material. In some examples, the stiffness of the grasped material may be determined from the jaw angle and/or separation and the applied force and/or torque determined during process 420. In some examples, one or more models (e.g., from the one or more models 190) may include one or more formulas, look-up tables, non-linear maps, and/or the like usable to determine material stiffness from the jaw angle and/or separation and the applied force and/or torque. In some examples, the one or more models used to determine stiffness of the grasped material may be determined from empirical studies, one or more machine learning mechanisms (e.g., one or more neural networks) trained based on test grasps of material with known stiffness, and/or the like.

In some examples, the one or more material characteristics may include a dielectric constant of the grasped material. In some examples, the dielectric constant of the grasped material may be determined from the jaw angle and/or separation determined during process 420 and the impedance of the grasped material determined during process 430. In some examples, the dielectric constant may be determined by delivering non-therapeutic energy to the material. In some examples, one or more models (e.g., from the one or more models 190) may include one or more formulas, look-up tables, non-linear maps, and/or the like usable to determine the dielectric constant of the grasped material from the jaw angle and/or separation and the impedance. In some examples, the one or more models used to determine the dielectric constant of the grasped material may be determined from empirical studies, one or more machine learning mechanisms (e.g., one or more neural networks) trained based on test grasps and/or energy delivery to material with known dielectric constant, and/or the like.

In some examples, the one or more material characteristics may include a desiccation level (e.g., moisture content) of the grasped material. In some examples, the desiccation level may provide an indicator of a level of current material sealing, an indication of whether the material is ready for cutting and/or sealing (e.g., it may be advantageous to squeeze moisture out the material by grasping before cutting and/or sealing). In some examples, the desiccation level may be determined from the jaw angle and/or separation, the applied force and/or torque, the applied pressure, the stiffness, the impedance, the dielectric constant, and/or the temperature for the grasped material. In some examples, one or more models (e.g., from the one or more models 190) may include one or more formulas, look-up tables, non-linear maps, and/or the like usable to determine the desiccation level of the grasped material from the jaw angle and/or separation, the applied force and/or torque, the applied pressure, the stiffness, the impedance, the dielectric constant, and/or the temperature. In some examples, the one or more models used to determine the dielectric constant of the grasped material may be determined from empirical studies, one or more machine learning mechanisms (e.g., one or more neural networks) trained based on test grasps and/or energy deliver to material with known desiccation level, and/or the like.

At a process 440, grasping and/or energy delivery by the tool is controlled based on the one or more grasp characteristics determined during process 420 and/or the one or more material characteristics determined during process 430 using one or more models, such as the one or more models 190. In some examples, the one or more grasp characteristics and/or the one or more material characteristics may be applied as inputs to the one or more models to determine one or more control parameters for controlling the grasping of the material and/or for controlling the energy delivery to the material. In some examples, the one or more control parameters may include one or more of a grasp set point (e.g., a grasp angle and/or separation), a rate of change in grasp set point (e.g., a grasping velocity), a force and/or torque set point, a force or torque set point, a current set point for one or more actuators used to actuate the jaws, a pressure set point, and/or the like.

In some examples, the one or more parameters for controlling energy delivery may include one or more of a voltage differential between a pair of electrodes, a current limit for energy delivery between a pair of electrodes, a target set point for material impedance, dielectric constant, and/or temperature indicative of successful sealing and/or cutting, an amount of sealing energy delivered to the grasped material, an amount of cutting energy delivered to the grasped material, and/or the like.

In some examples, the one or more control parameters for controlling the grasping of the material and/or the energy delivery to the material may include one or more thresholds for determining when to switch between control strategies, when to switch between different models, and/or the like.

According to some embodiments, a goal of using the one or more models is to implement grasping and/or energy delivery control strategies that reduce the likelihood of the material slipping during grasping and/or energy delivery, poor cutting of the grasped material, poor sealing of the grasped material, and/or the like. According to some embodiments, the one or more models may include models that provide guidance to one or more control strategies used for grasping and/or energy delivery that take advantage of shared knowledge and information between the grasping and the energy delivery control modules, such as grasp control module 170 and/or energy control module 180.

According to some embodiments, the one or more models may be used to control the amount of energy delivered based on one or more of the grasp characteristics determined during process 420. In some examples, the one or more models may indicate that the amount of energy to deliver may be related to the jaw angle and/or separation. In some examples, when the jaw angle and/or separation is larger, more energy is delivered because more material is grasped and when the jaw angle and/or separation is smaller, less energy is delivered because less material is grasped. In some examples, the relationship between the jaw angle and/or separation and energy to deliver may be one or more of linear, monotonic, subject to maximum and minimum energy delivery limits, and/or the like. In some examples, the one or more models may indicate that the amount of energy to deliver is inversely related to the rate of change in jaw angle and/or separation. In some examples, when the rate of change in jaw angle and/or separation is smaller, more energy is delivered to address stiffer and/or more slowly desiccating material and when the rate of change in jaw angle and/or separation is larger, less energy is delivered to address less stiff and/or more rapidly desiccating material. In some examples, the relationship between the rate of change jaw angle and/or separation and energy to deliver may be monotonic, subject to maximum and minimum energy delivery limits, and/or the like.

According to some embodiments, the one or more models may be used to determine how to control the grasping based on one or more of the material characteristics determined during process 430. In some examples, the one or more models may indicate that a force and/or torque limit of the grasping is inversely related to the impedance of the material. In some examples, when the impedance of the material is lower (e.g., its moisture content is higher and more desiccation is desired), the force and/or torque limit is raised for a stronger grasp that should help increase desiccation and when the impedance is higher, the force and/or torque limit is lowered as desiccation and/or sealing nears completion. In some examples, the relationship between the impedance and the force and/or torque limit may be one or more of monotonic, subject to maximum and minimum force and/or torque limits, and/or the like. In some examples, the one or more models may indicate that the force and/or torque limit is related to the rate of change in impedance. In some examples, when the rate of change in impedance is smaller (e.g., early in a cutting and sealing operation), the force and/or torque limit is increased to address stiffer and/or more slowly desiccating material, when the rate of change in impedance is larger (e.g., during the mid-portion of a cutting and sealing operation), the force and/or torque limit is lowered to address less stiff and/or more rapidly desiccating material, and when the rate of change in impedance is smaller (e.g., near the completion of a cutting and sealing operation), the force and/or torque limit is left unchanged and/or reduced. In some examples, the relationship between the rate of change in impedance and the force and/or torque limit may be subject to maximum and minimum force and/or torque limits, and/or the like.

According to some embodiments, the one or more models may be used to determine an amount of sealing energy to apply and an amount of cutting energy that are independently applied so as to achieve a desired ratio of sealing energy to cutting energy to improve the likelihood of a clean cut of the material with good sealing properties. In some examples, the one or more models may determine that a higher ratio of sealing energy to cutting energy is desirable when more compression of the material is desired, more desiccation of the material is desired, and/or greater material stiffness is detected, such as may be indicated by a higher jaw angle and/or separation, a lower rate of change in jaw angle and/or separation, a higher applied force and/or torque, a higher applied pressure, a lower rate of change in applied pressure, a higher rate of change in applied force and/or torque, a lower material impedance, a higher material temperature, a lower rate of change in material temperature and/or the like. In some examples, the one or more models may determine that a lower ratio of sealing energy to cutting energy when less compression of the material is desired, less desiccation of the material has to occur, and/or lesser material stiffness is detected, such as may be indicated by a lower jaw angle and/or separation, a higher rate of change in jaw angle and/or separation, a lower applied force and/or torque, a lower rate of change in applied force and/or torque, a higher applied pressure, a lower rate of change in applied pressure, a higher material impedance, a higher material temperature, a lower rate of change in material temperature, and/or the like. In some examples, the one or more models may indicate a higher ratio of sealing energy to cutting energy at the start of a cutting and sealing operation and a lower ratio of sealing energy to cutting energy at the end of the cutting and sealing operation. In some examples, the one or more models may implement the ratio of sealing energy to cutting energy by indicating that one or more of the current limits used to control the energy delivered by a pair of sealing electrodes and/or a pair of cutting electrodes be raised and/or lowered, that the voltage differential applied by the pair of sealing electrodes and/or the pair of cutting electrodes be raised and/or lowered (e.g., apply more cutting energy to the pair of sealing electrodes and/or apply more sealing energy to the pair of cutting electrodes).

According to some embodiments, the one or more models may be used to determine an ending condition that indicates when energy delivery is complete (e.g., when cutting and/or sealing is complete). In some examples, the one more models may receive as input any of the one or more grasp characteristics determined during process 420 and/or the one or more material characteristics determined during process 430 and determine one or more parameters that correspond to the ending condition. In some examples, the one or more parameters that correspond to the ending condition may include a threshold impedance of the grasped material, a threshold dielectric constant of the grasped material, a threshold temperature of the grasped material, and/or the like that indicates that sufficient energy has been delivered to the grasped material. In some examples, the threshold impedance may correspond to a minimum impedance that should be reached before energy delivery is complete. In some examples, the one or more models may indicate that the threshold impedance should increase when the one or more grasp characteristics and/or the one or more material characteristics indicate that progress of the grasp is slow (e.g., a higher jaw angle and/or separation, a lower rate of change in jaw angle and/or separation, a higher rate of change in applied force and/or torque, a higher applied pressure, a lower rate of change in applied pressure, a lower level of desiccation, a lower rate of change in desiccation, a higher material temperature, and/or the like) suggesting that a greater amount of material is being grasped, desiccation is slow, and/or the like.

According to some embodiments, the one or more models may indicate that energy delivery should be terminated. In some examples, the one or more models may indicate that energy delivery should be terminated when an ending condition is reached as discussed previously. In some example, the one or more models may indicate that energy deliver should be terminated when one or more of the grasp characteristics determined during process 420 and/or the one or more material characteristics determined during process 430 are outside of a desired range of values. In some examples, the desired range of values may correspond to a range of acceptable jaw angles and/or separations, a range of applied force and/or torque, a range of applied pressure, a range of material dielectric constants, a range of impedance, a range of material temperature, and/or the like and/or any combination of two or more characteristics outside of a respective desired range of values.

According to some embodiments, the one or more models may be used to replace a default energy delivery profile based on one or more of the grasp characteristics determined during process 420 and/or the one or more material characteristics determined during process 430. In some examples, the default energy delivery profile may be used when the grasp of the material is not difficult (e.g., the jaw angle and/or separation is below a threshold, the applied force and/or torque is below a threshold, the applied pressure is below a threshold, and/or the like) and the one or more models may be used when the grasp is difficult (e.g., the jaw angle and/or separation is above the threshold, the applied force and/or torque is above the threshold, the applied pressure is above the threshold, and/or the like).

The indications and/or outputs of the one or more models are then used to control the grasp and/or the energy delivery of the tool. In some examples, the indications and/or outputs are provided as parameters, thresholds, commands, and/or the like to the appropriate grasp control and/or energy delivery control algorithm, such as implemented by grasp control module 170 and/or energy control module 180. The grasp control and/or energy delivery control algorithms then provide one or more commands, signals, and/or the like to the systems for grasp and energy delivery, such as drive system 240 and/or energy system 260.

At a process 450, it is determined whether grasping and/or energy delivery should be stopped. In some examples, the determination may be based on when one or more of the grasp characteristics determined during process 420 and/or the one or more material characteristics determined during process 430 reach an ending condition as discussed above. When the ending condition is not reached and grasping and/or energy delivery should continue, processes 420-440 are repeated by returning to process 420. When the ending condition is reached and grasping and/or energy delivery should be stopped, the one or more models may be updated using optional process 460.

At optional process 460, the one or more models are updated. In some examples, the data collected during processes 420-440 (e.g., the one or more grasp characteristics, the one or more material characteristics, and/or the indications from the one or more models) may be used to update the one or more models based on the results of the grasp and/or energy delivery. In some examples, the data may be used as additional data points and/or training data usable to update the one or more models. In some examples, the additional data points may be used to update curve fitting, regression analyses, and/or the like that are the basis for the one or more models. In some examples, the additional training data may be used to update a machine learning system, such as a neural network, as additions to the supervised training data that is used in a back propagation training algorithm, a simulated annealing training algorithm, a stochastic gradient descent training algorithm, and/or the like.

According to some embodiments, whether or not the one or more models are updated by optional process 460, the one or more models may be used again by repeating method 400.

As discussed above and further emphasized here, FIG. 4 is merely an example which should not unduly limit the scope of the claims. One of ordinary skill in the art would recognize many variations, alternatives, and modifications. According to some embodiments, other factors may be considered during process 440 and provide one or more inputs to the one or more models used to control the grasping of and/or energy delivery to the grasped material. In some examples, the other factors may include one or more of operator preference, a known type of the grasped material, a type of procedure being performed on the grasped material, a type and/or a model of the tool used to grasp the material and deliver energy to the grasped material, and/or the like. In some examples, the other factors may include calibration parameters for the tool that is stored in the tool and/or stored in a database that may account for variations among tools, wear and/or change in the tool over one or more uses, and/or the like.

According to some embodiments, process 440 may be adapted to provide additional information on grasping, cutting, and/or sealing to an operator. In some examples, the additional information may include a prediction of whether cutting and/or sealing are likely to be successful, a recommended delay time with additional grasping before cutting and/or sealing should begin, an estimated amount of time before cutting and/or sealing are complete, and/or the like.

Accordingly to some embodiments, method 400 may be used with other energy modalities than the electric and/or radio frequency modality discussed primarily with respect to FIG. 4. In some examples, the other energy modalities may include on or more of ultrasonic, magnetic, thermal, light, and/or the like.

According to some embodiments, method 400 may be adapted for other energy delivery applications other than energy delivery via one or more pairs of cutting and/or sealing electrodes. In some examples, the other energy deliver applications may include cutting and sealing using a single pair of electrodes, ablation, cutting with an ultrasonic scalpel, and/or the like. According to some embodiments, method 400 may be used where sealing is performed using energy delivery and cutting is performed via a mechanical cutting element, such as a knife.

According to some embodiments, method 400 may be adapted to support calibration of tools. In some examples, one or more of the parameters of the one or models used during process 440 and optionally updated during process 460 may be stored in a memory located in the tool and/or in a database that may be queried based on a identifier of the tool so as to support customization of the one or more models for each individual tool to be used during method 400. In some examples, the one or more parameters may include one or more coefficients, one more control points for curve and/or function modeling, one or more neural weights and/or biases, and/or the like. In some examples, the one or more parameters for a tool may be initially calibrated at manufacturing time by using the tool to grasp and apply energy to one or more materials with known properties (e.g., size, stiffness, dielectric constant, and/or the like) and using the test grasps and/or energy deliveries to customize the one or more parameters based on differences between the actual performance of the tool and the performance indicated by the one or more models. In some examples, the one or more parameters may be further updated before each use by using the tool to grasp and deliver energy to one or more materials with known properties. In some examples, the updating of the one or more models during process 460 may be used to update the one or more parameters.

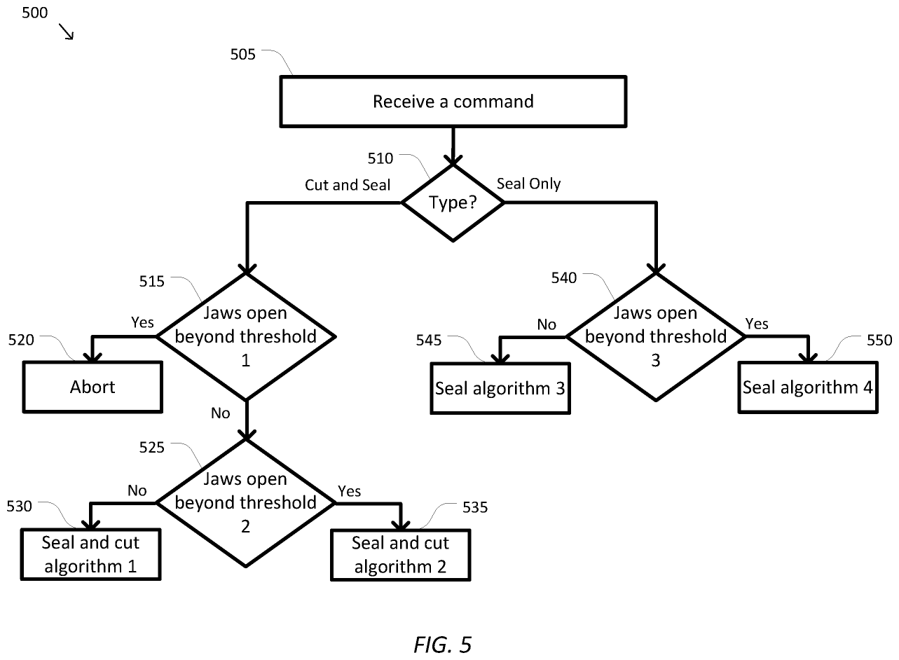

FIG. 5 is a simplified diagram of a method 500 for energy delivery according to some embodiments. One or more of the processes 505-550 of method 500 may be implemented, at least in part, in the form of executable code stored on non-transitory, tangible, machine readable media that when run by one or more processors (e.g., the processor 150 in control unit 140) may cause the one or more processors to perform one or more of the processes 505-550. In some embodiments, method 500 may be performed by one or more modules, such as grasp control module 170 and/or energy control module 180. In some embodiments, portions of method 400 associated with grasping (e.g., sensing of mechanical and/or kinematic information and mechanical control of grasping jaws) may be performed by grasp control module 170 and portions of method 400 associated with energy delivery (e.g., sensing of electrical properties and controlling of energy delivery) may be performed by energy control module 180 with grasp control module 170 and energy control module 180 cooperating to share sensor and control information so as to optimize energy delivery to a grasped material. According to some embodiments, method 500 may be consistent with processes 420-450 of method 400.

At a process 505, a command is received. In some examples, the command may be received from an operator. In some examples, the command may be received as a result of a user interface activation, activation of one or more buttons, switches, levers, and/or the like, a voice command, and/or the like. In some examples, the command may be a command to seal only or a command to cut and seal with a different user interface control, button, switch, lever, voice command, and/or the like being used to indicate the type of the command.

At a process 510, a type of the command is determined. When the type of the command is a cut and seal command, the cut and seal command is further processed beginning with a process 515. When the type of the command is a seal only command, the seal only command is further processed beginning with a process 540.

At the process 515, it is determined whether jaws of an energy delivery device are grasping a material with an opening greater than a configurable first threshold. In some examples, each of the jaws may be consistent with jaw 300. In some examples, the first threshold may correspond to a jaw angle, a jaw separation, and/or the like between the jaws. In some examples, the first threshold may correspond to a jaw opening that indicates that more material is being grasped than can be cut and/or sealed. When the opening is greater than the first threshold, the cutting and sealing operation is aborted using a process 520. When the opening is not greater than the first threshold, the opening is further analyzed beginning with a process 525.

At the process 520, the cutting and sealing operation is aborted and no energy is delivered to the material. In some examples, an alert and/or notification may be provided to the operator indicating that too much material is grasped for proper cutting and/or sealing. In some examples, the alert may include one or more of a visual alert (e.g., a blinking light, a color change, a textual message, and/or the like), an audio alert (e.g., a beep, a series of beeps, a tone, a voice instruction, and/or the like), haptic feedback, and/or the like. Method 500 then concludes or alternatively returns to process 505 to await an additional command.

At the process 525, it is determined whether the jaws of the energy delivery device are grasping the material with an opening greater than a configurable second threshold smaller than the first threshold. In some examples, the second threshold may correspond to a jaw angle, a jaw separation, and/or the like between the jaws. In some examples, the second threshold may correspond to a jaw opening that indicates that more material is grasped than is ideal for cutting and/or sealing. When the opening is not greater than the second threshold, the cutting and sealing operation continues beginning with a process 530. When the opening is greater than the second threshold, the cutting and sealing operation continues beginning with a process 535.

At the process 530, a first seal and cut procedure is applied. In some examples, the first seal and cut procedure may begin with the delivery of sealing energy using one or more sealing electrodes. In some examples, the sealing energy may be delivered for a first configurable period of time, until an impedance of the material falls below a first threshold, and/or the like. In some examples, the first threshold may be between 200 and 600 ohms when the material is anatomic tissue. In some examples, the first threshold may correspond to a target desiccation level. In some examples, the impedance may be determined indirectly based on an amount of current through the one or more sealing electrodes. In some examples, when the impedance of the material reaches the first threshold, and/or the first configurable period of time expires, cutting energy may also be delivered by one or more cutting electrodes and/or a mechanical cutting may be actuated to cut the material. In some examples, the sealing energy and/or the cutting energy may continue to be delivered until the impedance of the material rises above a second threshold and/or a second configurable period of time has elapsed. In some examples, the second threshold may be between 200 and 600 ohms when the material is anatomical tissue. In some examples, the second period of time may be 10 seconds.

In some examples, the first seal and cut procedure may be aborted if the impedance of the material does not rise above the second threshold before the second time period elapses. In some examples, the first seal and cut procedure may be aborted if the impedance of the material is above a third threshold. In some examples, the third threshold may be 1000 ohms when the material is anatomical tissue. In some examples, an alert and/or notification may be provided to the operator indicating that cutting and/or sealing was not successfully completed due to high impedance in the material and/or expiration of the second time period. In some examples, the alert may include one or more of a visual alert (e.g., a blinking light, a color change, a textual message, and/or the like), an audio alert (e.g., a beep, a series of beeps, a tone, a voice instruction, and/or the like), haptic feedback, and/or the like.

In some examples, one or more of the impedance thresholds, time periods, amount of energy to deliver, and/or the like may be selected based on one or more of a type of the material, a procedure being performed, operator preference, and/or the like.

Once process 530 is complete and/or is aborted, method 500 then concludes or alternatively returns to process 505 to await an additional command.

At the process 535, a second seal and cut procedure is applied. In some examples, process 535 may include one or more of increasing an amount of sealing energy and/or cutting energy delivered relative to the first seal and cut procedure of process 530, increasing an amount of time that sealing energy and/or cutting energy is delivered relative to the first seal and cut procedure of process 530, changing a sealing energy waveform and/or a cutting energy waveform relative to the sealing energy waveform and/or the cutting energy waveform of the first seal and cut procedure of process 530, and/or the like. In some examples, the second seal and cut procedure may use similar impedance and/or timing tests as used by the first seal and cut procedure of process 530 in order to determine whether successful sealing and/or cutting or unsuccessful sealing and/or cutting is obtained. Method 500 then concludes or alternatively returns to process 505 to await an additional command.

At the process 540, it is determined whether the jaws of the energy delivery device are grasping the material with an opening greater than a configurable third threshold. In some examples, the third threshold may correspond to a jaw angle, a jaw separation, and/or the like between the jaws. In some examples, the third threshold may correspond to a jaw opening that indicates more material is being grasped than can be adequately sealed. In some examples, the third threshold is equal to the first threshold. When the opening is not greater than the third threshold, the opening is further analyzed beginning with a process 545. When the opening is greater than the third threshold, the sealing operation continues beginning with a process 550.

At the process 545, a third seal procedure is applied. In some examples, the third seal procedure may begin with the delivery of sealing energy using the one or more sealing electrodes. In some examples, the sealing energy may be delivered for a third configurable period of time, until an impedance of the material falls below a fourth threshold, and/or the like. In some examples, the fourth threshold may be between 200 and 600 ohms when the material is anatomic tissue. In some examples, the fourth threshold may correspond to a target desiccation level. In some examples, the impedance may be determined indirectly based on an amount of current through the one or more sealing electrodes. In some examples, when the impedance of the material reaches the fourth threshold, and/or the third configurable period of time expires, a fourth configurable time period may begin with sealing energy still be delivered by the one or more sealing electrodes. In some examples, the sealing energy may continue to be delivered until the impedance of the material rises above a fifth threshold and/or the fourth period of time has elapsed. In some examples, the fifth threshold may be between 200 and 600 ohms when the material is anatomical tissue. In some examples, the fourth period of time may be 10 seconds.

In some examples, the third seal procedure may be aborted if the impedance of the material does not rise above the fifth threshold before the fourth time period elapses. In some examples, the third seal procedure may be aborted if the impedance of the material is above a sixth threshold. In some examples, the sixth threshold may be 1000 ohms when the material is anatomical tissue. In some examples, an alert and/or notification may be provided to the operator indicating that cutting and/or sealing was not successfully completed due to high impedance in the material and/or expiration of the fourth time period. In some examples, the alert may include one or more of a visual alert (e.g., a blinking light, a color change, a textual message, and/or the like), an audio alert (e.g., a beep, a series of beeps, a tone, a voice instruction, and/or the like), haptic feedback, and/or the like.

In some examples, one or more of the impedance thresholds, time periods, amount of energy to deliver, and/or the like may be selected based on one or more of a type of the material, a procedure being performed, operator preference, and/or the like.

Once process 545 is complete and/or is aborted, method 500 then concludes or alternatively returns to process 505 to await an additional command.

At the process 550, a fourth seal procedure is applied. In some examples, process 550 may include one or more of increasing an amount of sealing energy delivered relative to the third seal procedure of process 545, increasing an amount of time that sealing energy is delivered relative to the third seal procedure of process 545, changing a sealing energy waveform relative to the sealing energy waveform of the third seal procedure of process 545, and/or the like. In some examples, the fourth seal procedure may use similar impedance and/or timing tests as used by the third seal procedure of process 545 in order to determine whether successful sealing or unsuccessful sealing is obtained. Method 500 then concludes or alternatively returns to process 505 to await an additional command.

As discussed above and further emphasized here, FIG. 5 is merely an example which should not unduly limit the scope of the claims. One of ordinary skill in the art would recognize many variations, alternatives, and modifications. According to some embodiments, method 500 may include more than the three depicted thresholds used to measure jaw opening. In some examples, any number of thresholds may be used to create a corresponding number of separate seal and cut and/or seal algorithms, with each of the seal and cut and/or seal algorithms using their own combination of sealing energy delivered, cutting energy delivered, a time that sealing energy is delivered, a time that cutting energy is delivered, a sealing energy waveform, a cutting energy waveform, impedance thresholds, timing thresholds, and/or the like.

In some examples, the thresholds, time periods, and/or the like may be omitted with one or more functions based on jaw opening being used to determine one or more of the sealing energy delivered, cutting energy delivered, the time that sealing energy is delivered, the time that cutting energy is delivered, parameters of the sealing energy waveform, parameters of the cutting energy waveform, impedance thresholds, time periods, and/or the like.

Some examples of control units, such as control unit 140 may include non-transitory, tangible, machine readable media that include executable code that when run by one or more processors (e.g., processor 150) may cause the one or more processors to perform the processes of method 400. Some common forms of machine readable media that may include the processes of method 400 are, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and in a manner consistent with the scope of the embodiments disclosed herein.

1. A computer-assisted device comprising:
an end effector having a first jaw, a second jaw, and a plurality of electrodes for delivering energy; and
one or more processors coupled to the end effector, the one or more processors being configured to:
grasp a material using the first jaw and the second jaw;
determine one or more characteristics of the grasp;
determine one or more characteristics of the material; and
control one or more of the grasp or energy delivery by the plurality of electrodes based on the determined one or more characteristics of the grasp and the determined one or more characteristics of the material.

2. The computer-assisted device of claim 1, wherein:
the end effector is part of a medical device; and
the material is anatomical tissue.

3-6. (canceled)

7. The computer-assisted device of claim 1, wherein the end effector further comprises a cutting mechanism controlled by the one or more processors.

8. The computer-assisted device of claim 1, wherein the end effector includes one or more sensors.

9. The computer-assisted device of claim 1, wherein the one or more characteristics of the grasp include one or more of:
a pressure applied to the material by the first and second jaws;
a rate of change in the pressure applied to the material by the first and second jaws;
an angle between the first and second jaws;
a rate of change in the angle between the first and second jaws;
a separation between the first and second jaws;
a rate of change in separation between the first and second jaws;
a force or torque applied by the first and second jaws to the material;
a rate of change in the force or torque applied by the first and second jaws to the material;
a force or torque applied by an actuator used to actuate one or both of the first or second jaws;
a rate of change in the force or torque applied by the actuator; or
an amount of articulation of an articulated wrist coupling the end effector to a shaft.

10. The computer-assisted device of claim 1, wherein the one or more characteristics of the material include one or more of:
a temperature of the material;
a rate of change in the temperature of the material;
a stiffness of the material;
a rate of change in the stiffness of the material.
an impedance of the material;
a rate of change in the impedance of the material;
a dielectric constant of the material; or
a rate of change in the dielectric constant of the material.

11-12. (canceled)

13. The computer-assisted device of claim 1, wherein to control the grasp or the energy delivery, the one or more processors are configured to:
provide one or more of the characteristics of the grasp or one or more of the characteristics of the material as inputs to one or more models; and
use one or more outputs of the one or more models as parameters to the controlling.

14-17. (canceled)

18. The computer-assisted device of claim 1, wherein to control the energy delivery, the one or more processors are configured to control an amount of energy or amount of time energy is delivered based on one or more of the characteristics of the grasping, an energy delivery profile, or both the one or more characteristics of the grasping and the energy delivery profile.

19. (canceled)

20. The computer-assisted device of claim 1, wherein to control the energy delivery, the one or more processors are configured to control an amount of sealing energy delivered and an amount of cutting energy delivered independently.

21. (canceled)

22. The computer-assisted device of claim 1, wherein to control the grasp or the energy delivery, the one or more processors are configured to determine an ending condition for stopping the grasp or the energy delivery.

23-24. (canceled)

25. The computer-assisted device of claim 1, wherein the one or more processors are further configured to switch between a default energy delivery profile and an energy delivery profile determined using one or more models based on one or more of the characteristics of the grasp or the characteristics of the material.

26. (canceled)

27. A method comprising:
grasping, by one or more processors, a material using a first jaw and a second jaw of an end effector;
determining, by the one or more processors, one or more characteristics of the grasping;
determining, by the one or more processors, one or more characteristics of the material; and
controlling, by the one or more processors, one or more of the grasping or energy delivery by a plurality of electrodes of the end effector based on the determined one or more characteristics of the grasp and the determined one or more characteristics of the material.

28-34. (canceled)

35. The method of claim 27, wherein the one or more characteristics of the grasping include one or more of:
a pressure applied to the material by the first and second jaws;
a rate of change in the pressure applied to the material by the first and second jaws;
an angle between the first and second jaws;
a rate of change in the angle between the first and second jaws;
a separation between the first and second jaws;
a rate of change in separation between the first and second jaws;
a force or torque applied by the first and second jaws to the material;
a rate of change in the force or torque applied by the first and second jaws to the material;
a force or torque applied by an actuator used to actuate one or both of the first or second jaws;
a rate of change in the force or torque applied by the actuator; or
an amount of articulation of an articulated wrist coupling the end effector to a shaft.

36. The method of claim 27, wherein the one or more characteristics of the material include one or more of:
a temperature of the material;
a rate of change in the temperature of the material;
a stiffness of the material;
a rate of change in the stiffness of the material.
an impedance of the material;
a rate of change in the impedance of the material;
a dielectric constant of the material; or
a rate of change in the dielectric constant of the material.

37-43. (canceled)

44. The method of claim 27, wherein controlling the energy delivery comprises controlling an amount of energy or amount of time energy is delivered based on one or more of the characteristics of the grasping, an energy delivery profile, or both the one or more characteristics of the grasping and the energy delivery profile.

45. (canceled)

46. The method of claim 27, wherein controlling the energy delivery comprises controlling an amount of sealing energy delivered and an amount of cutting energy delivered independently.

47-50. (canceled)

51. The method of claim 27, further comprising switching between a default energy delivery profile and an energy delivery profile determined using one or more models based on one or more of the characteristics of the grasping or the characteristics of the material.

52. (canceled)

53. A non-transitory machine-readable medium comprising a plurality of machine-readable instructions which when executed by one or more processors are adapted to cause the one or more processors to perform a method comprising:
grasping a material using a first jaw and a second jaw of an end effector;
determining one or more characteristics of the grasping;
determining one or more characteristics of the material; and
controlling one or more of the grasping or energy delivery by a plurality of electrodes of the end effector based on the determined one or more characteristics of the grasp and the determined one or more characteristics of the material.

54. The non-transitory machine-readable medium of claim 53, wherein the one or more characteristics of the grasping include one or more of:
a pressure applied to the material by the first and second jaws;
a rate of change in the pressure applied to the material by the first and second jaws;
an angle between the first and second jaws;
a rate of change in the angle between the first and second jaws;
a separation between the first and second jaws;
a rate of change in separation between the first and second jaws;
a force or torque applied by the first and second jaws to the material;
a rate of change in the force or torque applied by the first and second jaws to the material;
a force or torque applied by an actuator used to actuate one or both of the first or second jaws;
a rate of change in the force or torque applied by the actuator; or
an amount of articulation of an articulated wrist coupling the end effector to a shaft.

55. The non-transitory machine-readable medium of claim 53, wherein the one or more characteristics of the material include one or more of:
a temperature of the material;
a rate of change in the temperature of the material;
a stiffness of the material;
a rate of change in the stiffness of the material.
an impedance of the material;
a rate of change in the impedance of the material;
a dielectric constant of the material; or
a rate of change in the dielectric constant of the material.
